(19) United States
(12) Patent Application Publication
(10) Pub. No.: US 2008/0312722 A1
(43) Pub. Date: Dec. 18, 2008

(54) HEAT STORAGE/RELEASE DEVICE

(76) Inventor: Wei-Hsun WANG, Yung-Ho City (TW)

Correspondence Address: SINORICA, LLC, 528 FALLSGROVE DRIVE, ROCKVILLE, MD, 20850, US

(21) Appl. No.: 12/138,581

(22) Filed: Jun. 13, 2008

(30) Foreign Application Priority Data
Jun. 14, 2007 (TW) 096209769

(51) Int. Cl. A61F 7/00

(52) U.S. Cl. 607/96

(57) ABSTRACT

A heat storage/release device for hot/cold compress application is disclosed to include a bag, which defines therein a sealed accommodation chamber, a heat storage/release material, which is accommodated in the accommodation chamber of the bag and adapted for storing/releasing heat energy, and flexible members fastened to the bag at two opposite sides relative to the accommodation chamber and bendable to hold the bag in a predetermined shape and to secure the bag to a person's face.

### BACKGROUND OF THE INVENTION

1. Field of the Invention

The present invention relates to a heat storage/release device and more particularly, to such a heat storage/release device that can be secured on a person's face to perform hot or cold compression without any fastening band.

2. Description of the Related Art

Many heat storage/release devices are known and commercially available to keep the body warm, or for the purpose of cold compress. Conventionally, a hot water bladder may be used for keeping the body warm. There are also hot compress bag that comprises a bag body sealed with fluid, a metal actuator strip mounted in the bag body to actuate the fluid, causing chemical reaction to release heat. During the heat producing and releasing procedure, the hot compress bag provides heat energy for hot compress application.

Nowadays, women or girls may take plastic surgery so as to have a better looking. After the plastic surgery, the face usually would need a cold compress for 48 hours to stop the bleeding and swelling. After 48-hour cold compress, a hot compress will be needed to eliminate the inflammation and disperse swelling. Furthermore, when a nasal allergy syndrome appeared, a hot compression is usually taken to smoothen the syndrome.

A conventional heat storage/release device for hot/cold compress purpose may be provided or used with a fastening band so that the heat storage/release device can be secured on a user's face or nose for hot/cold compress. Besides the inconvenience of using fasten band to tie a heat storage/release device on an user's face or nose the use of a fastening band also obstructs the sense of beauty.

Therefore, it is desirable to provide a heat storage/release device that eliminates the aforesaid drawbacks.

### SUMMARY OF THE INVENTION

The present invention has been accomplished under the circumstances in view. Therefore, the main object of the present invention is to provide a heat storage/release device, which can conveniently fastened to a person's face for hot/cold compress, without any fastening band. The other object of the present invention, the heat storage/release device is reusable.

To achieve the objects of the present invention, the heat storage/release device, is for hot/cold compress application, comprising a bag, which defines therein a sealed accommodation chamber, a heat storage/release material, which is accommodated in the accommodation chamber of the bag and adapted for storing/releasing heat energy, and at least one flexible member fastened to at least one of the two opposite lateral sides of the bag and bendable to hold the bag in a predetermined shape and to secure the bag on a person's face.

### BRIEF DESCRIPTION OF THE DRAWINGS

FIG. 1 is a schematic drawing showing a heat storage/release device in accordance with a first embodiment of the present invention.

FIG. 2 is a schematic drawing showing a heat storage/release device in accordance with a second embodiment of the present invention.

FIG. 3 is a schematic drawing showing a heat storage/release device in accordance with a third embodiment of the present invention.

FIG. 4 is a schematic drawing showing of the third embodiment of the present invention, showing the heat storage/release device bent into a curved shape for application.

### DETAILED DESCRIPTION OF THE PREFERRED EMBODIMENT

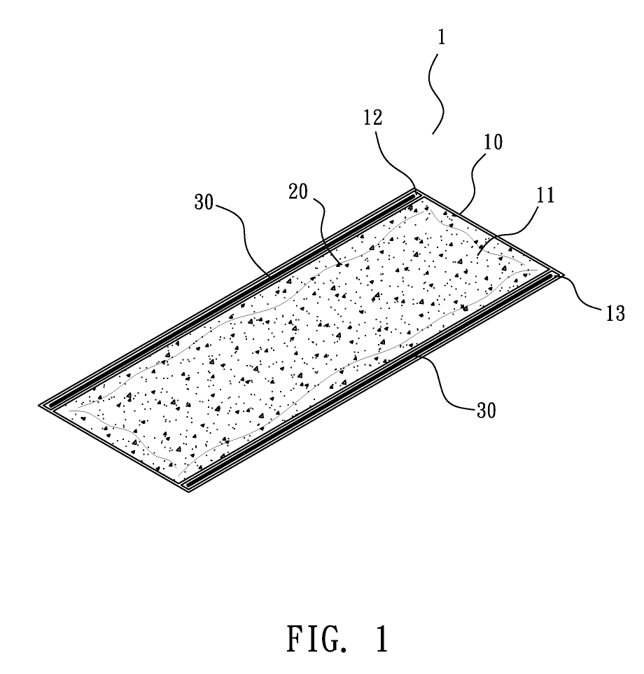

Referring to FIG. 1, a heat storage/release device 1 in accordance with a first embodiment of the present invention is shown comprising a bag 10, a heat storage/release material 20, and at least one flexible member 30.

The bag 10 is a container with a predetermined shape, for example, rectangular or circular shape. According to this embodiment, the bag 10 has a rectangular shape. The bag 10 defines therein a sealed accommodation chamber 11, which accommodates the heat storage/release material 20. The bag 10 is made of, but not limited to, polymeric material. Furthermore, the bag 10 is preferably a transparent bag. For the purpose of being reusable, the material of the bag 10 has durable and extensible characteristics and a certain thickness. During fabrication, high frequency sealing technique or any other suitable sealing technique is employed to seal the periphery of the bag 10.

The bag 10 comprises two side chambers 12 and 13 arranged at two opposite lateral sides relative to the sealed accommodation chamber 11 and extending along the length of the rectangular bag 10 for accommodating the at least one flexible member 30. High frequency sealing technique or any other suitable sealing technique is applied to the bag 10, forming the side chambers 12 and 13.

The heat storage/release material 20 has the power of storing/releasing heat energy, and is accommodated in the sealed accommodation chamber 11 of the bag 10. The heat storage/release material 20 can be pure water, saturated sodium acetate solution, or any suitable known fluid that storages and releases heat.

According to the present preferred embodiment, the number of the at least one flexible member 30 is 2, and the two flexible members 30 are respectively sealed to the inside of the side chambers 12 and 13 of the bag 10. Therefore, the user can bend the flexible members 30 into a curved status, thereby securing the heat storage/release device 1 to a person's face to perform a hot or cold compress. Therefore, the heat storage/release device 1 eliminates the drawbacks of conventional designs.

The flexible member 30 can be a metal wire rod made of iron, tin, lead or copper; or a metal wire rod with a flexible tube that is sleeved onto the metal wire rod. The flexible tube can be a plastic or fabric tube.

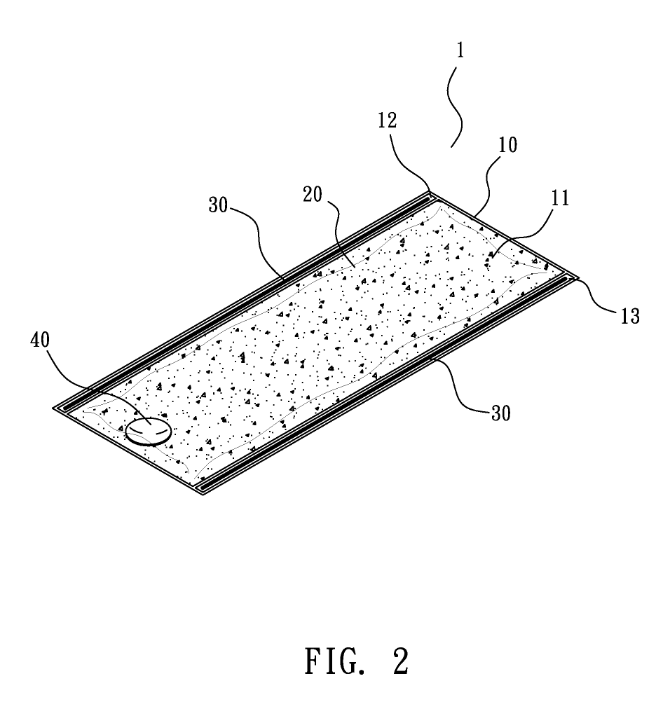

FIG. 2 illustrates a heat storage/release device 1 in accordance with a second embodiment of the present invention. This second embodiment is substantially similar to the aforesaid first embodiment with the exception that this second embodiment comprises an actuator 40 affixed to the inside of the sealed accommodation chamber 11 of the bag 10. The actuator 40 can be a metal strip that produces microwaves to actuate the heat storage/release material 20 when bent in one direction, causing the heat storage/release material 20 to release heat. In this case, the heat storage/release material 20 can be saturated sodium acetate solution.

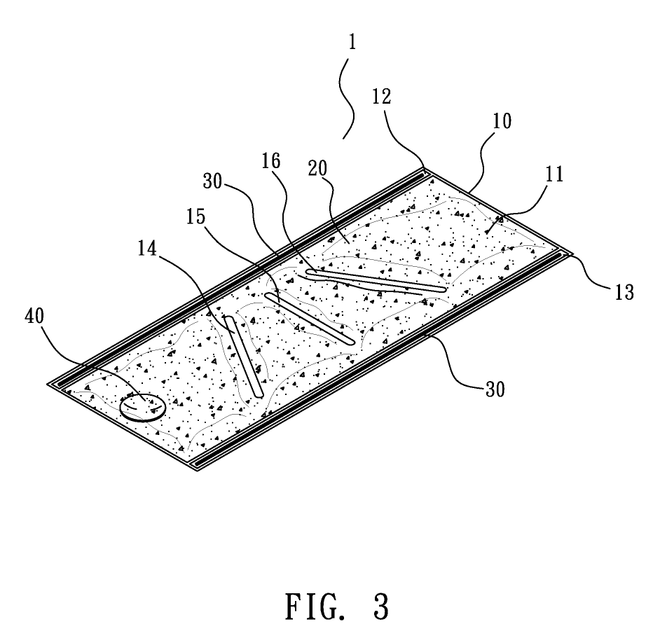

FIG. 3 illustrates a heat storage/release device 1 in accordance with a third embodiment of the present invention. This third embodiment is substantially similar to the aforesaid second embodiment with the exception that the bag 10 of this third embodiment has a first seal line 14, a second seal line 15 and a third seal line 16. The second seal line 15 transversely seals the middle of the bag 10, dividing the sealed accommodation chamber 11 of the bag 10 into two symmetrical halves. The first seal line 14 and the third seal line 16 obliquely seals the bag 10 and disposed at two opposite sides relative to the second seal line 15, diving each half of the sealed accommodation chamber 11 into two sub chambers (not indicated in the drawing). However, the first seal line 14, the second seal line 15 and the third seal line 16 are kept apart from the side chambers 12 and 13 at a predetermined distance so that the sub chambers are in fluid communication with one another. This embodiment reduces the flowing speed of the heat storage/release material 20 in the sealed accommodation chamber 11. The seal lines 14, 15 and 16 are made to fit the configuration of a person's nose, enabling the heat storage/release device 1 to be positively secured to a person's nose.

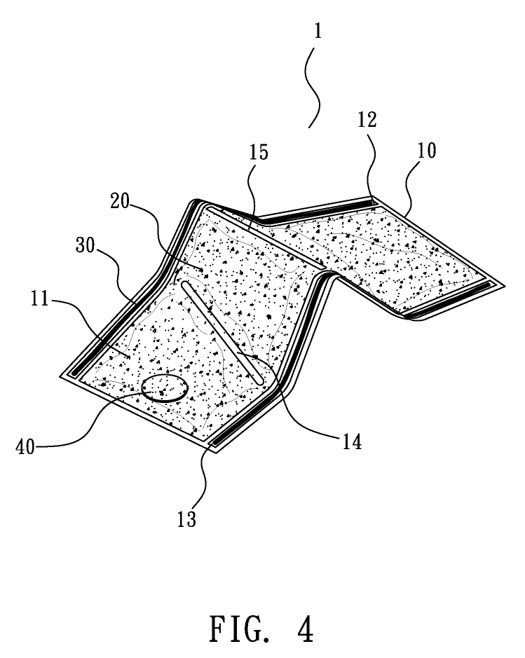

Referring to FIG. 4, the flexible members 30 can be bent into a curved shape so that the heat storage/release device 1 can be positively secured to a person's nose to apply a hot compress or cold compress to the nose. After the application, the flexible members 30 are reversely bent and return to their former straight shape, allowing the heat storage/release device 1 for a repeat use.

By means of the aforesaid design, the heat storage/release device 1 is reusable, and can conveniently be fastened to a person's face directly for hot or cold compress without the use of any fastening band. Therefore, the invention eliminates the drawbacks of conventional designs.

Although particular embodiments of the invention have been described in detail for purposes of illustration, various modifications and enhancements may be made without departing from the spirit and scope of the invention.

What is claimed is:

1. A heat storage/release device for hot/cold compress application, comprising:
a bag, said bag defining therein a sealed accommodation chamber;
a heat storage/release material accommodated in the accommodation chamber of said bag and adapted for storing/releasing heat energy; and
at least one flexible member fastened to at least one of two opposite lateral sides of said bag and bendable to hold said bag in a predetermined shape.

2. The heat storage/release device as claimed in claim 1, wherein said heat storage/release material is water.

3. The heat storage/release device as claimed in claim 1, wherein said bag is made of a polymeric material.

4. The heat storage/release device as claimed in claim 1, wherein said heat storage/release material is saturated sodium acetate solution.

5. The heat storage/release device as claimed in claim 1, further comprising an actuator made of a metal strip and fixedly mounted in said accommodation chamber inside said bag for causing said saturated sodium acetate solution to make a chemical reaction.

6. The heat storage/release device as claimed in claim 1, wherein said bag further comprises two longitudinal side chambers extending along two opposite lateral sides of said accommodation chamber, and at least one flexible member mounted in at least one of said longitudinal side chambers and bendable to hold said bag in a predetermined shape.

7. The heat storage/release device as claimed in claim 6, wherein the number of said at least one flexible member is two, and the two flexible members are respectively mounted in said two longitudinal side chambers and made by high frequency sealing.

8. The heat storage/release device as claimed in claim 7, wherein said two flexible members could be a metal or a metal covered with a plastic layer, respectively, wherein the flexible members are made of metal.

9. The heat storage/release device as claimed in claim 8, wherein said metal wire is made of one of the material group of iron, tin, lead and copper, and said flexible covering is made of a plastic material.

10. The heat storage/release device as claimed in claim 6, wherein said bag further comprises a middle seal line that divides said accommodation chamber into two symmetrical half chambers that are in fluid communication with each another, and two oblique seal lines spaced from said middle seal line at two opposite sides and dividing each said half chamber into two sub chambers that are in fluid communication with each other.

11. The heat storage/release device as claimed in claim 10, wherein said middle seal line and said oblique seal lines are formed in said bag by means of high frequency sealing or heat fusing.
